(19) United States
(12) Patent Application Publication
(10) Pub. No.: US 2018/0153411 A1
(43) Pub. Date: Jun. 7, 2018

(54) Hybrid IR-US RTLS System

(71) Applicant: CenTrak, Inc., Newtown, PA (US)

(72) Inventors: Israel Amir, Newtown, PA (US); Tal Singer, Newtown, PA (US)

(21) Appl. No.: 15/832,393

(22) Filed: Dec. 5, 2017

Related U.S. Application Data
(60) Provisional application No. 62/497,852, filed on Dec. 5, 2016.

(51) Int. Cl. A61B 5/00; A61B 8/00; A61B 8/08

(52) U.S. Cl. CPC A61B 5/0091; A61B 5/0035; A61B 8/4416; A61B 8/5238; A61B 8/4483

(57) ABSTRACT

A hybrid infrared-ultrasound real time location system includes at least one base station having an infrared emitter and a plurality of ultrasound emitters and at least one tag. The tag receives an infrared signal from the infrared emitter and ultrasound signals from the ultrasound emitters and a time difference is determined between the time-of-arrival of the IR signal and the time-of-arrival of each ultrasound signal. Based on the time relationship of the respective transmissions of the IR signals and the US signals, the tag can measure the respective time-of-flight of each of the US transmissions from the US emitters to the tag and compute the distance from the base-station.

### STATEMENT OF RELATED CASES

This case claims priority of U.S. provisional patent application 62/497,852, which was filed Dec. 5, 2016 and is incorporated by reference herein.

### FIELD OF THE INVENTION

This invention pertains generally to indoor real time location systems.

### BACKGROUND OF THE INVENTION

Indoor Real Time Location Systems (RTLS) are prevalent today more than ever. Their accuracy has tremendously improved and they have shown substantial ROI.

Current RTLS typically utilize a secondary technology, in addition to the traditional RF-based methods that use trilateration and signatures to provide location. Those two techniques are not accurate enough to provide room and sub-room (e.g., hospital bed) level accuracy.

The two main secondary technologies used today are infrared (IR) and ultrasound (US). Ultrasound has the advantage of time of flight feature and in some cases, can provide very high-accuracy, while IR has an inherent power consumption advantage due to shorter signals that both the transmitter and receiver must process, transmit, and receive. These signals are, in most cases, the IDs associated with the transmitter and received by the receiver.

### SUMMARY OF THE INVENTION

The present invention provides a new approach to RTLS wherein IR is used for ID communications and US is used for “delineation” information.

In accordance with the illustrative embodiment, a direct measurement of the time-of-flight from an emitter to a RTLS tag or other device for which an estimation of location and/or height is required (hereinafter collectively referred to as a “tag”). Most current methods use differential time-of-arrival as the direct estimation of the actual time-of-flight, which is not practical using only US. In accordance with the present teachings, an estimate is obtained of the time difference between the time-of-arrival of the IR signal (i.e., essentially immediate as it travels at the speed of light) and the time-of-arrival of the US signal, which propagates at a speed of about 300 meters/second in air.

It is assumed that all base-stations and ultrasound emitters are synchronized, such that they possess the same time of origin. But unless otherwise noted, this does not mean that the signals are transmitted at the same time; again, it means that the clocks are synchronized. There are multiple methods for synchronization, as well known to those skilled in the art, and such methods are not described herein to keep the focus on subject matter that is germane to the invention.

In some embodiments, an IR emitter (e.g., a base station) transmits a periodic IR beacon with multiple associated US emitters that transmit US signals with a known time relationship to the IR transmissions. A tag includes both IR receiver and US receivers so that it can receive both the IR signal and the multiple US signals. Based on the time relationship of the respective transmissions of the IR signals and the US signals, the tag can measure the respective time-of-flight of each of the US transmissions from the US emitters to the tag and compute the distance from the base-station. The method “works” because IR signals propagate at the speed of light, they can be assumed to cross the distance between the IR emitter and the tag's IR receiver at “zero” time relative to US, which propagates at 300 meters per second.

### BRIEF DESCRIPTION OF THE DRAWINGS

FIG. 1 depicts an illustrative embodiment of an integrated device including two US transducers and an IR emitter in accordance with the present teachings.

FIG. 2 depicts an illustrative embodiment of an integrated device including three US transducers and an IR emitter in accordance with the present teachings.

FIG. 3A depicts an illustrative embodiment of an integrated device showing how US transducers 1 and 2 create arbitrary virtual walls orthogonal to the line connecting the two US transducers.

FIG. 3B depicts an illustrative embodiment of an integrated device showing how US transducers 3 and 4, which are disposed orthogonally to transducers 1 and 2 of FIG. 3A, create arbitrary virtual walls orthogonal to the line connecting US transducers 3 and 4.

FIG. 3C depicts an illustrative embodiment of an integrated device showing virtual walls resulting from the use of four US transducers.

FIG. 3D depicts an illustrative embodiment of an integrated device showing virtual walls resulting from a minimal configuration of three US transducers.

### DETAILED DESCRIPTION

FIG. 1: Height X Deciphered from Known H1 and the Calculated h2: X=H1−h2.

The manner in which the two US emitters are set (i.e., vertically) enables the estimation of height anywhere in the area under coverage. The estimation holds anywhere around the vertical axis.

In some embodiments, the IR signal carries also an ID that allows the tag to recognize the room it is in. Likewise, the US signals can carry the room ID. Limiting the transmission of the room ID to IR (for example) makes both the US emitter (for example) much more efficient than having to transmit the ID on both US and IR.

In some embodiments, the IR transmissions are synchronized with tag IR reception such that the tag knows when to turn on the IR receiver and the US receivers in order to reduce power consumption. For example, if the room under coverage is 15 ft×15 ft, and the tag knows when the IR was transmitted and when the US signals were transmitted, the tag would need to turn on the IR receiver for the duration of the IR signal and the US receivers for about 15-20 milliseconds to capture the US signals, as the speed of propagation of the US signals is known and the maximum time for them to reach the US receiver is calculable.

In some embodiments, the IR emitter and the US emitter are integrated into a single device. The integrated device can be mounted on the wall and the two US emitters are set at the two edges of the IR emitter to make the vertical distance between them as large as possible and thus improve the height-estimation accuracy.

X, Y and X,Y,Z Position Estimation.

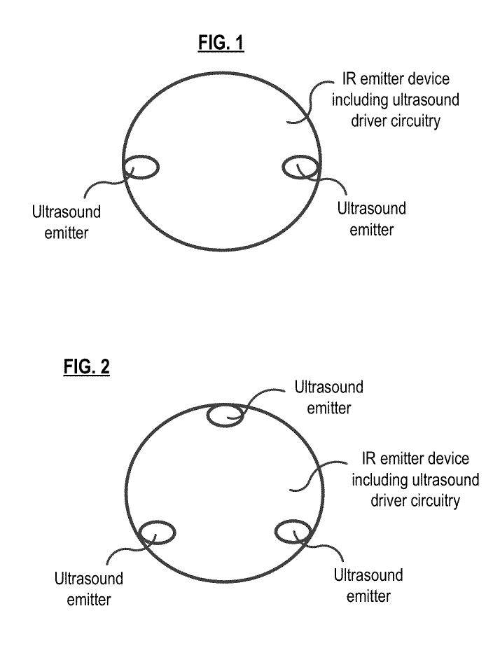

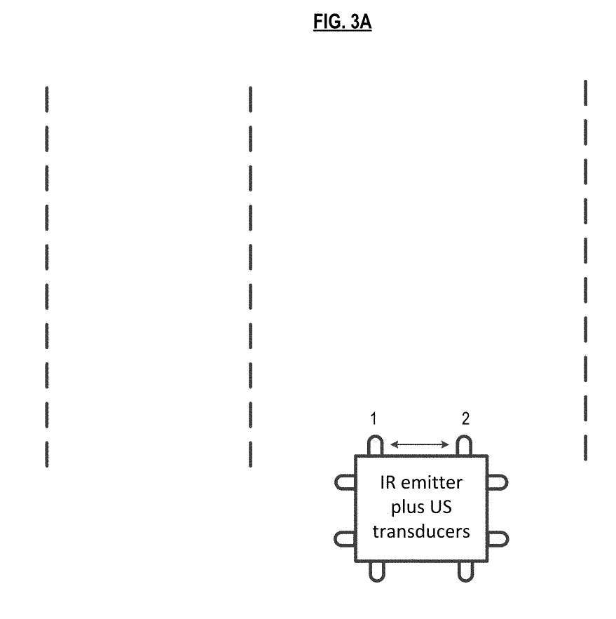

It can be shown that using three US emitters, instead of two as in the embodiment above, wherein the three US emitters are position such that they do not fall on a line, provides pinpoint accuracy in the X, Y and Z axis. See FIG. 2. If, for example, three US emitters are placed on a ceiling, the position of a tag can be estimated using the three different distances from the three emitters to the tag.

Finding the Closest US Emitter.

In areas in which there is more than one US emitter, it is possible to find out which emitter is closest to the tag using synchronization. The method is discussed below for embodiments in which there are two emitters and three emitters; the method is scalable to any number of US emitters.

The US emitters need to be synchronized. If a tag knows when the US emitters transmit, the tag can determine which is closest. If the US emitters transmit at the same time, then signal that is received first by the tag is sourced from the closest US emitter.

To avoid collisions, the US signals from multiple US emitters are transmitted with a slight time offset so that the source of each signal is clear to the tag's receiver. The time offset needs to consider the size of the zone to be covered and the time it takes for the US signals (typically short bursts) associated with each US transducer to “die” or use a coded message to allow the US signals to be deciphered in parallel.

The main advantage of the first approach is that generally, it is more power efficient. The main drawback of the first approach is that a tradeoff must be struck between the time between US burst transmissions and the maximum speed that the tag can move before impacting accuracy performance. But it can be shown that for people and asset tracking, 30 milliseconds between bursts can support tag speeds of up to 2-3 meter/second without significantly impacting accuracy performance.

What is described above pertains to a single base station; that is, it is the “building block” of the present system and method. We now extend the system to multiple base stations and multiple IR emitters (which can be pointing at different zones), each having multiple associated US emitters, as described previously.

In some embodiments, all the base-stations are synchronized such that the US emitters must be synchronized with each other. This is particularly advantageous when two different IR emitters are designed to transmit the same IR identification, which is often done to increase the size of zone. In some other embodiments, two close IR emitters (with their US emitters) transmit the same IR-ID for the purpose of defining a left and right boundary to a zone.

In some embodiments, there are two base stations, each having a different IR-ID and associated ID and which are placed “back-to-back” such that the IR emitters send their respective IDs to opposite directions. Each one of those emitters have associated US emitters sending US signals essentially to the same direction as the IR emitters. In some embodiments, the IR emitters transmit their associated transmissions about every 30 milliseconds. In some other embodiments, the first US burst associated with the first IR emitter transmit its burst at the same time the IR signal is transmitted and the second burst at about 30 milliseconds afterwards. The second IR emitter transmits its IR-ID 30 milliseconds after the first IR-ID is transmitted, with the associated US bursts following the same pattern as the first base station.

In some additional embodiments, the US bursts can be transmitted at different times than the IR signals, with the caveat that the time offset is known to the tag. With such knowledge, the tag can decipher which burst belong to which IR-ID and the time between US bursts associated with the same IR-ID stays small and is known to the tag. This type of approach not only enables the determination of which side of the back-to-back pair the tag is located, but also where in the zone the tag is based on simple triangulation of the ranges calculated from each US transducer to the tag.

In yet some further embodiments, two base stations are placed near each other and pointing in essentially the same direction, each base station having at least one US emitter. In some embodiments, a tag can decipher on which side of the line to either left or right of the two base-stations, the tag is based on comparing the time-of-arrival of the signals from the US transducers, the one on the left and the one on the right. This can also be achieved with a single IR base-base station having two US transducers, but we add the embodiment of different IR-IDs with two base-stations to explain how this is accomplished in more complex embodiments, wherein multiple virtual lines can create “virtual” rooms.

In some additional embodiments, a single IR emitter with at least three US emitters is used to map an entire area and create an arbitrary division of zones. In this embodiment, the tag receives a single IR signal carrying the IR-ID of the base-station and estimates the exact location of the tag in 3D space. The tag will send to the system, using RF, the IR-ID with the three distances from the three US emitters. The server maps the three distances into a virtual zone. Back-to-back base stations can be used to help alleviate range and distance inaccuracies.

Those skilled in the art will appreciate that this approach is readily scalable using a synchronized base-station. For example, assume that each one of multiple base stations transmit their IR-ID and US bursts in a periodic manner, wherein the period is large enough to ensure that US bursts from one base station will not be confused with the US bursts of another base-station. It turns out that 40 milliseconds is enough time for an US burst to die, so a period of 40 milliseconds is expected to be sufficient to result in a clear association of the received US bursts with the associated IR.

In some other embodiments, differential time-of-arrival for the US pulses is used. Assume that the US emitters' cycle in a known order (if they do not carry their own ID). The relative differential time-of-arrival is measured for each possible pair, and subtracting the known heartbeat rate will yield the closest emitter.

If, for example, the heartbeat is one second, we will have TOA of T1, T2, and T3 from, say, three emitters. The differences D1,2−1, D2,3−1 and D1,3−2 are calculated. The reason for the “−2” (seconds) is because the transmission between the first and the third pulses was set to exactly two seconds. Assume that D1,2−1 is larger than 0. This means that the US emitter 1 is closer to the tag then US emitter 2. D1,3−2 must be tested. If D1,3−2 is, for example, less than 0, it means that US emitter 3 is closer to the tag than US emitter 1. Therefore, US emitter 3 is the closest US emitter to the tag.

Dealing with Secondary Reflections and Loss of Signals.

It is possible to lose a signal. It is also possible to miss a direct US signal and receive only secondary reflections. Also, because the US signals are not emanating from the exact location, it is possible that one of the US bursts will be direct and the other missing or a result of a secondary reflection.

In some embodiments, the received signal strength indication (RSSI) of the received US bursts are used decide if one of the bursts is from a direct transmission and one is from a secondary reflection. In some embodiments, such an RSSI discrepancy will cause the receiver to drop the measurement.

In some other embodiments, if the time difference between the US bursts associated with the same IR emitter exceeds the associated distance between the US transducers, it indicates that one of the bursts is a secondary reflection. In some embodiments, that will prompt the tag to drop the measurement.

Practical Zone Mapping into Subzones.

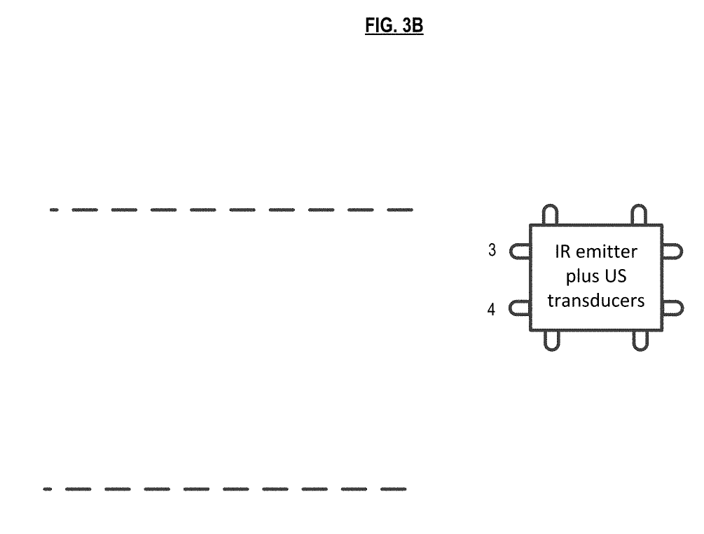

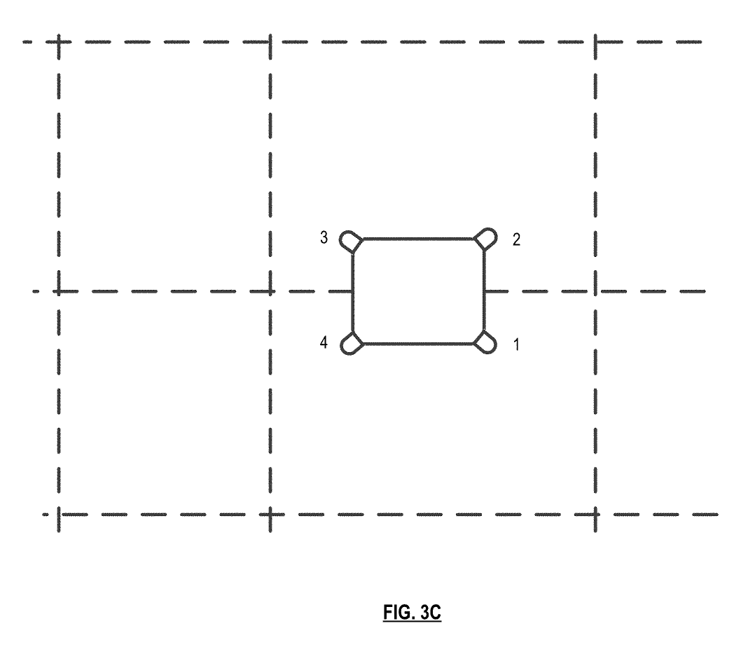

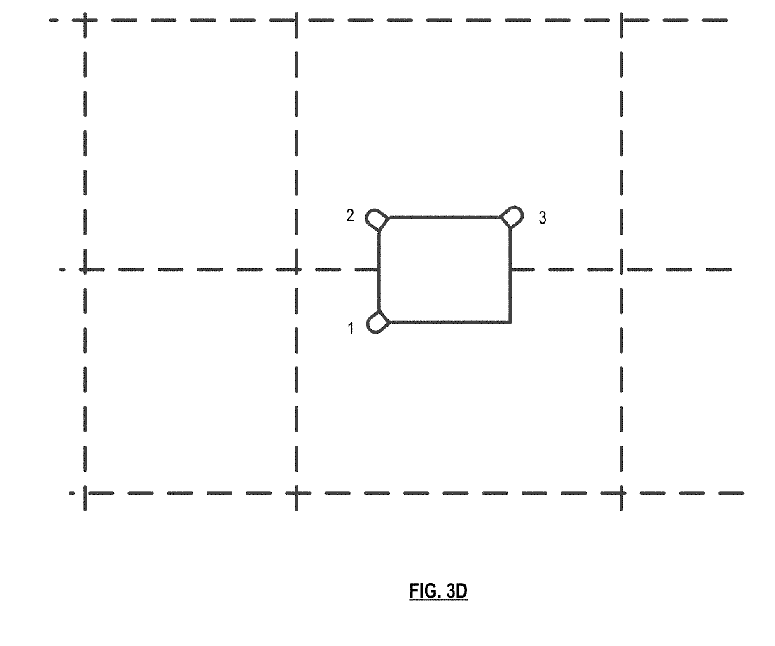

To ensure that sufficiently strong US signals arrive at the receiver, in one embodiment, the base station includes a single IR emitter and eight associated US transmitters, wherein two US transmitters are on each side of a square housing to address all four orientations. In this embodiment, following the IR-ID emission, each side, in turn in a cycle, transmits two US bursts to define a “virtual wall” in the direction orthogonal to the line connecting the center of these two transducers. This is illustrated in FIGS. 3A and 3b.

Using this technique, many virtual walls can be created. Specifically, the tag sends to the server both the IR-ID and the two time-of-arrivals associated with the two transducers relative to the IR emission. Based on programmed settings, the server can then decide to which orthogonal zone the tag belongs. Likewise, two other US transducers set on another side of the housing of the base station can create virtual walls (in the orthogonal direction to those two transducers).

In some embodiments, fewer transducers can be used (i.e., as few as three as shown in FIG. 3D), but it is often important for the main lobe of the transmission from the US transducers to be narrow with high gain. This increases the probability of direct reception by the tag versus secondary reflections from walls and other objects, which can cause signal loss at best and substantial errors at worst. It is well known that US signals can reach an US receiver by “refraction” through different modes of US waves, and the difference between direct line-of-sight and refracted light is not large.

Even if a tag is facing away from the emitting base-station, the US signals will reach the tag's receiver with only slightly longer travel and not necessarily from reflection of nearby objects. Hence the necessity for sufficiently strong US signals. Thus, even in situations in which there is no direct line-of-sight and the signals are weakened, the tag's receiver will still be capable of receiving the US signals. Clearly, the transmitted power can be increased, but this is problematic for battery-powered base stations. In RTLS, the base station most often be battery powered for reasons of cost. The gain from directional transducers typically outweighs the penalty of increasing the number of transmissions. For example, transducer gain can be as large as 10-20 dB higher when directional.

IR/US Equivalency for ID Communication.

In this specification, the functions of the IR emitter and the US emitter can be interchanged. That is, US can provide the same information provided by IR except for the length measurement from the time difference between the IR and US time-of-arrival. But, as will be clear to one skilled in the art, an additional US transducer can resolve this issue using differential times of arrival.

It is to be understood that the disclosure describes a few embodiments and that many variations of the invention can easily be devised by those skilled in the art after reading this disclosure and that the scope of the present invention is to be determined by the following claims.

What is claimed:

1. A method for determining a location of a tag in a real time location system, the method comprising:
receiving, at the tag, an infrared signal a base station;
receiving, at the tag, multiple ultrasound signals from the base station;
determining a time difference, for each ultrasound signal, between a time-of-arrival at the tag of the infrared signal and a time-of-arrival of the ultrasound signal; and
computing a distance from the tag to the base station based on the propagation speed through air of ultrasound signals.
